(19) United States
(12) Patent Application Publication
(10) Pub. No.: US 2026/0325743 A1
(43) Pub. Date: Oct. 22, 2026

(54) Anti-Self Sealing Element Integrated in Flexible Reservoir Bag and Method of Manufacturing the Same

(71) Applicant: Becton, Dickinson and Company, Franklin Lakes, NJ (US)

(72) Inventors: Steve Beguin, Nenzlingen (CH); Kieran Kelly, Dublin (IE); Stuart Plascott, Foxrock (IE); Christian Sandmann, Wayne, NJ (US)

(21) Appl. No.: 19/110,516

(22) PCT Filed: Sep. 22, 2023
(86) PCT No.: PCT/US2023/033439

§ 371 Date: Mar. 11, 2025

Related U.S. Application Data
(60) Provisional application No. 63/409,375, filed on Sep. 23, 2022.

(51) Int. Cl. A61M 5/142; A61M 5/148

(52) U.S. Cl. CPC A61M 5/14248; A61M 5/148; A61M 2005/14252; A61M 2207/00

(57) ABSTRACT

A drug delivery device configured to deliver a medicament to a patient includes a cannula for piercing the patient's skin, a pump for driving medicament to the cannula, and a reservoir bag fluidly connected to the pump. The reservoir bag includes a first membrane layer and a second membrane layer defining a medicament chamber. At least one of the first membrane layer and the second membrane layer comprises a tunnel extending at least partially into the medicament chamber.

### CROSS-REFERENCE TO RELATED APPLICATION

The present application claims priority to U.S. Provisional Application Ser. No. 63/409,375 entitled “Anti-Self Sealing Element Integrated in Flexible Reservoir Bag and Method of Manufacturing the Same”, the entire disclosure of which is hereby incorporated by reference.

### BACKGROUND OF THE DISCLOSURE

### Field of the Invention

The present disclosure relates generally to devices, systems, and methods for the delivery of pharmaceutical compositions to a patient. More particularly, the present disclosure relates to an anti-self-sealing element for a reservoir bag for an injector, tooling for forming such a reservoir bag, and associated methods.

### Description of Related Art

Wearable medical devices, such as automatic injectors, have the benefit of providing therapy to the patient at a location remote from a clinical facility and/or while being worn discretely under the patient's clothing. On a physician's determination, a wearable medical device can be applied to the patient's skin and configured to automatically deliver a dose of the pharmaceutical composition within a predetermined time period after applying the wearable medical device to the patient's skin, such as after a 27-hour delay. After the device delivers the pharmaceutical composition to the patient, the patient may subsequently remove and dispose of the device.

Conventional automatic injectors typically include an internal reservoir bag in which the medicament is contained. Under certain circumstances, particularly when the reservoir bag is nearly empty, opposing sides of the reservoir bag can be drawn together under the suction of the pump of the injector. This can partially or wholly seal the outlet of the reservoir bag and prevents intended delivery of the medicament.

Accordingly, there is a need for automatic injectors with features to prevent unintentional self-sealing.

### SUMMARY OF THE DISCLOSURE

Embodiments of the present disclosure are directed to a drug delivery device configured to deliver a medicament to a patient. The drug delivery device includes a cannula for piercing the patient's skin, a pump for driving medicament to the cannula, and a reservoir bag fluidly connected to the pump. The reservoir bag includes a first membrane layer and a second membrane layer defining a medicament chamber. At least one of the first membrane layer and the second membrane layer includes a tunnel extending at least partially into the medicament chamber.

In some embodiments, the reservoir bag is an internal reservoir bag contained within an internal region of the drug delivery device.

In some embodiments, the first membrane layer is asymmetric relative to the second membrane layer.

In some embodiments, the first membrane layer is symmetric relative to the second membrane layer.

In some embodiments, a surface area of the second membrane layer is greater than a surface area of the first membrane layer.

In some embodiments, a surface area of the second membrane layer is approximately the same as a surface area of the first membrane layer.

In some embodiments, the tunnel has a raised profile.

In some embodiments, the tunnel defines a flow channel in fluid communication with a port of the reservoir bag.

In some embodiments, the tunnel extends an entire length of the medicament chamber.

In some embodiments, the tunnel terminates at an intermediate location within the medicament chamber.

In some embodiments, the tunnel is sufficiently strong to maintain shape under vacuum produced by the pump.

In some embodiments, the tunnel prevents the first and second membrane layers from laying entirely flat against one another.

Other embodiments of the present disclosure are directed to a vacuum molding device for forming a reservoir bag of a drug delivery device. The vacuum molding device includes a die defining a cavity, and a vacuum channel extending from the cavity. The cavity corresponds to a profile of a tunnel to be formed in the internal reservoir bag. The vacuum channel is configured to draw at least one of a first membrane layer and a second membrane layer of the reservoir bag into the cavity.

In some embodiments, the vacuum molding device further includes a sealing plate configured to join the first membrane layer to the second membrane layer.

In some embodiments, the sealing plate includes one or more prongs configured to join the first membrane layer to the second membrane layer.

In some embodiments, the sealing plate is configured to be moved toward the die.

In some embodiments, the die includes one or more passageways through which one or more profiles of the sealing plate extend.

Other embodiments of the present disclosure are directed to a pinch-forming device for forming a reservoir bag of a drug delivery device. The pinch-forming device includes a pair of pinching grips configured for engaging a second membrane layer of the reservoir bag on opposite sides of a tunnel to be formed in the second membrane layer, and an actuator configured for drawing the pinching grips toward one another to form the tunnel in the second membrane layer. A first membrane layer of the reservoir bag remains stationary as the actuator draws the pinching grips toward one another.

In some embodiments, the actuator includes a cylinder.

In some embodiments, the pinch-forming device further includes a sealing plate configured to join the first membrane layer to the second membrane layer.

In some embodiments, the sealing plate includes one or more prongs configured to join the first membrane layer to the second membrane layer.

Other embodiments of the present disclosure are directed to a method of forming a reservoir bag of a drug delivery device. The method includes positioning a first membrane layer and a second membrane layer on a die, the die defining a cavity corresponding to a profile of a tunnel to be formed in the internal reservoir bag, applying a vacuum to the cavity to draw the second membrane layer into the cavity, and joining the first membrane layer to the second membrane layer to lock the tunnel in place.

In some embodiments, joining the first membrane layer to the second membrane layer includes engaging at least one of the first membrane layer and the second membrane layer with a heated sealing plate.

In some embodiments, the die includes one or more passageways through which the one or more profiles of the heated sealing plate extend.

Other embodiments of the present disclosure are directed to a method of forming a reservoir bag of a drug delivery device. The method includes engaging a second membrane layer of the reservoir bag with a pair of pinching grips, drawing the pinching grips toward one another to pinch the second membrane layer to form a tunnel in the membrane layer, and joining a first membrane layer to the second membrane layer to lock the tunnel in place.

In some embodiments, joining the first membrane layer to the second membrane layer includes engaging at least one of the first membrane layer and the second membrane layer with a heated sealing plate.

Other embodiments of the present disclosure are directed to a reservoir bag for storing a medicament. The reservoir bag includes a first membrane layer and a second membrane layer. The first membrane layer and the second membrane layer define a medicament chamber. At least one of the first membrane layer and the second membrane layer includes a tunnel extending at least partially into the medicament chamber. The first membrane layer is asymmetric relative to the second membrane layer.

In some embodiments, a surface area of the second membrane layer is greater than a surface area of the first membrane layer.

In some embodiments, a surface area of the second membrane layer is approximately the same as a surface area of the first membrane layer.

In some embodiments, the tunnel has a raised profile.

In some embodiments, the tunnel defines a flow channel in fluid communication with a port of the reservoir bag.

In some embodiments, the tunnel extends an entire length of the medicament chamber.

In some embodiments, the tunnel terminates at an intermediate location within the medicament chamber.

In some embodiments, the tunnel is sufficiently strong to maintain shape under vacuum produced by a pump of a drug delivery device to which the reservoir bag is connected.

In some embodiments, the tunnel prevents the first and second membrane layers from laying entirely flat against one another.

Other embodiments of the present disclosure are directed to a filling station for filling a drug delivery device configured to deliver a medicament to a patient. The filling station includes an injector socket for receiving the drug delivery device, the drug delivery device including a reservoir bag receiving medicament, a cartridge socket for receiving a refill reservoir bag containing the medicament, and a pump configured to deliver the medicament from the refill reservoir bag to the reservoir bag of the drug delivery device. The refill reservoir bag includes a first membrane layer and a second membrane layer defining a medicament chamber. At least one of the first membrane layer and the second membrane layer includes a tunnel extending at least partially into the medicament chamber.

In some embodiments, the first membrane layer is asymmetric relative to the second membrane layer.

In some embodiments, the first membrane layer is symmetric relative to the second membrane layer.

In some embodiments, a surface area of the second membrane layer is greater than a surface area of the first membrane layer.

In some embodiments, a surface area of the second membrane layer is approximately the same as a surface area of the first membrane layer.

In some embodiments, the tunnel has a raised profile.

In some embodiments, the tunnel defines a flow channel in fluid communication with a port of the refill reservoir bag.

In some embodiments, the tunnel extends an entire length of the medicament chamber.

In some embodiments, the tunnel terminates at an intermediate location within the medicament chamber.

In some embodiments, the tunnel is sufficiently strong to maintain shape under vacuum produced by the pump.

In some embodiments, the tunnel prevents the first and second membrane layers from laying entirely flat against one another.

These and other features and characteristics of drug delivery devices, reservoir bags, devices for manufacturing the same, drug delivery device filling stations, and associated methods will become more apparent upon consideration of the following description and the appended claims with reference to the accompanying drawings, all of which form a part of this specification, wherein like reference numerals designate corresponding parts in the various drawings. It is to be expressly understood, however, that the drawings are for the purpose of illustration and description only.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a perspective view of a drug delivery device according to an embodiment of the present disclosure;

FIG. 2 is a cross-sectional view of the drug delivery device, taken along section line A-A of FIG. 1;

FIG. 3 is a perspective view of the drug delivery device of FIG. 1, with the main cover and reservoir removed for clarity;

FIG. 4 is a partial bottom view of the drug delivery device of FIG. 1;

FIG. 5 is a partial cross-sectional view of the drug delivery device, taken along section line B-B of FIG. 4;

FIG. 6 is a fluid path schematic diagram of the drug delivery device of FIG. 1;

FIG. 7 is a schematic view of a reservoir bag of the drug delivery device of FIG. 1;

FIG. 8 is a partial cross-sectional view of the reservoir bag of FIG. 7;

FIG. 9 is cross-sectional view of a vacuum molding device for forming the reservoir bag of FIG. 7, according to an embodiment of the present disclosure;

FIG. 10 is cross-sectional view of a vacuum molding device for forming the reservoir bag of FIG. 7, according to an embodiment of the present disclosure;

FIG. 11 is cross-sectional view of a vacuum molding device for forming the reservoir bag of FIG. 7, according to an embodiment of the present disclosure;

FIG. 12 is cross-sectional view of a pinch-forming device for forming the reservoir bag of FIG. 7, in a first position, according to an embodiment of the present disclosure;

FIG. 13 is cross-sectional view of the pinch forming device of FIG. 12, in a second position;

FIG. 14 is a partial cross-sectional view of the reservoir bag of FIG. 7;

FIG. 15 is a top view of the reservoir bag of FIG. 14;

FIG. 16 is a perspective view of a filling station for a drug delivery, according to an embodiment of the present disclosure;

FIG. 17 is a schematic diagram of a transfer tray of the filling station of FIG. 16, loaded with an on-body injector and a refill cartridge; and

FIG. 18 is a fluid path diagram of the transfer tray of FIG. 17;.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION

Spatial or directional terms, such as “left”, “right”, “inner”, “outer”, “above”, “below”, and the like, are not to be considered as limiting as the invention can assume various alternative orientations.

All numbers used in the specification and claims are to be understood as being modified in all instances by the term “about”. By “about” is meant a range of plus or minus ten percent of the stated value. As used in the specification and the claims, the singular form of “a”, “an”, and “the” include plural referents unless the context clearly dictates otherwise.

The terms “first”, “second”, and the like are not intended to refer to any particular order or chronology, but instead refer to different conditions, properties, or elements. By “at least” is meant “greater than or equal to”. By “not greater than” is meant “less than or equal to”.

As used herein, the term “at least one of” is synonymous with “one or more of”. For example, the phrase “at least one of A, B, and C” means any one of A, B, and C, or any combination of any two or more of A, B, and C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C. Similarly, as used herein, the term “at least two of” is synonymous with “two or more of”. For example, the phrase “at least two of D, E, and F” means any combination of any two or more of D, E, and F. For example, “at least two of D, E, and F”′ includes one or more of D and one or more of E; or one or more of D and one or more of F; or one or more of E and one or more of F; or one or more of all of D, E, and F.

It is also to be understood that the specific devices and processes illustrated in the attached drawings, and described in the following specification, are simply exemplary examples of the disclosure. Hence, specific dimensions and other physical characteristics related to the examples disclosed herein are not to be considered as limiting.

As used herein, the terms “fluidly connected”, “in fluid communication”, and derivatives thereof may be used interchangeably to mean a connection between a first component and a second component that allows fluids to flow from the first component to the second component and vice versa.

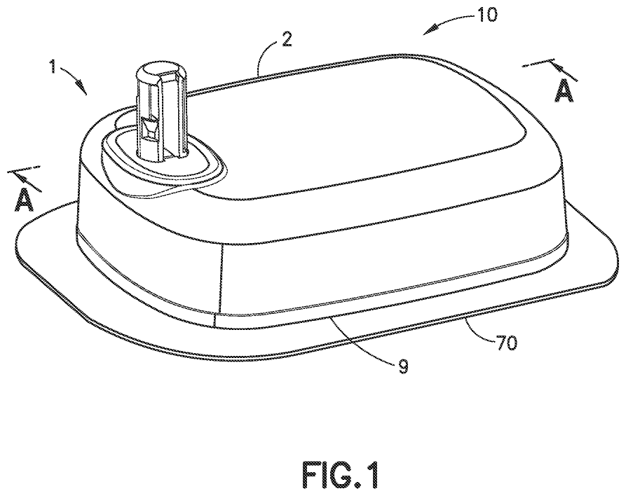

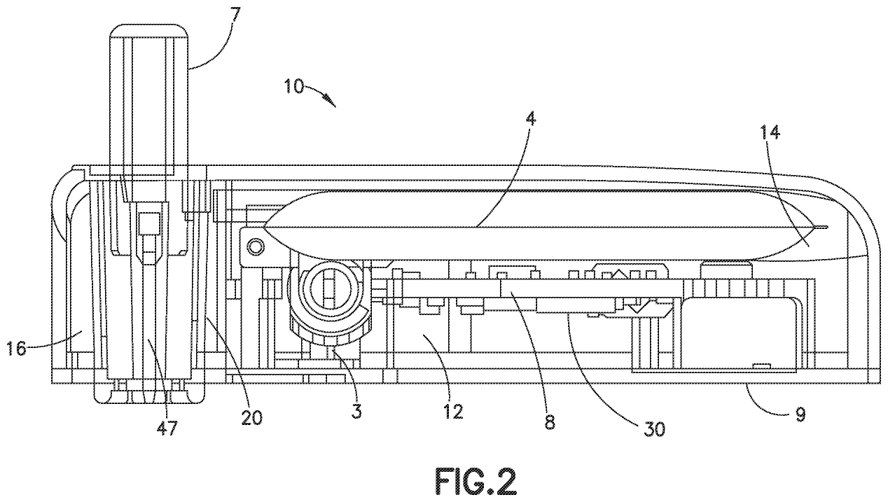

Referring first to FIGS. 1 and 2, a drug delivery device 1 is illustrated in accordance with an embodiment of the present disclosure. The drug delivery device 1 generally includes components for piercing a patient's skin and delivering medicament to the patient. In particular, the drug delivery device 1 includes a housing 10 having a main cover 2 liquid-sealed and/or hermetically sealed to a base 9. The base 9 houses and supports various components as described herein. The hermetic seal between the main cover 2 and the base 9 prevents fluids and particles from the outside environment from contaminating the internal components of the housing 10. In some embodiments, the drug delivery device 1 may also include a vent or a vent membrane to provide pressure equalization within the housing 10. The base 9 may include an adhesive pad 70 for securing the drug delivery device 1 to the patient.

FIG. 2 is a cross-sectional view of the drug delivery device 1 illustrating various internal components. The main cover 2 and the base 9 define an interior 12 which may be divided by a barrier 20 into a first internal region 14 and a second internal region 16. The drug delivery device 1 includes a reservoir bag 4 for storing a medicament (such as insulin), a pump 3 for pumping the medicament out of the internal reservoir bag 4, and a force sensing resistor 30 for detecting an amount of pressure in a medicament flow path. The drug delivery device 1 also includes electronics 8 for programming and operating the drug delivery device 1, and an insertion mechanism 7 for inserting a cannula 47 into a skin of the patient to deliver medicament. In the embodiment illustrated in the FIG. 2, the reservoir bag 4 is an internal reservoir bag that is contained within the interior 12 of the housing 10. In other embodiments, the reservoir bag 4 may be an external reservoir bag that is located outside the housing 10 and connected to the cannula 47 via tubing.

With continued reference to FIG. 2, the first internal region 14 of the interior 12 of the drug delivery device 1 includes components such as the pump 3, the force sensing resistor 30, and the electronics 8. Examples of the electronics 8 include semiconductor chips, controllers, diodes, antennas, coils, batteries, discrete components (resistors and capacitors, for example) and circuit boards used to operate and control the pump 3 and other electromechanical components of the drug delivery device 1. Due to the sensitive nature of the electronics 8, the first internal region 14 may be hermetically sealed and/or liquid-tight to prevent fluid ingress from the external environment.

With continued reference to FIG. 2, the second internal region 16 may include the insertion mechanism 7 and the cannula 47. In some embodiments, because the insertion mechanism 7 interfaces with the skin of a patient, the second internal region 16 is neither a hermetically sealed environment, nor a liquid-tight environment.

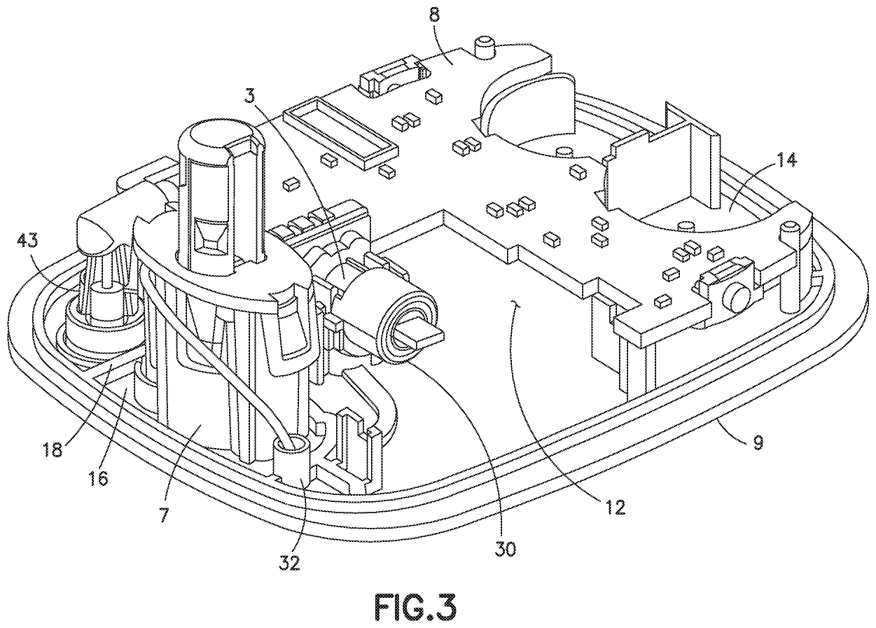

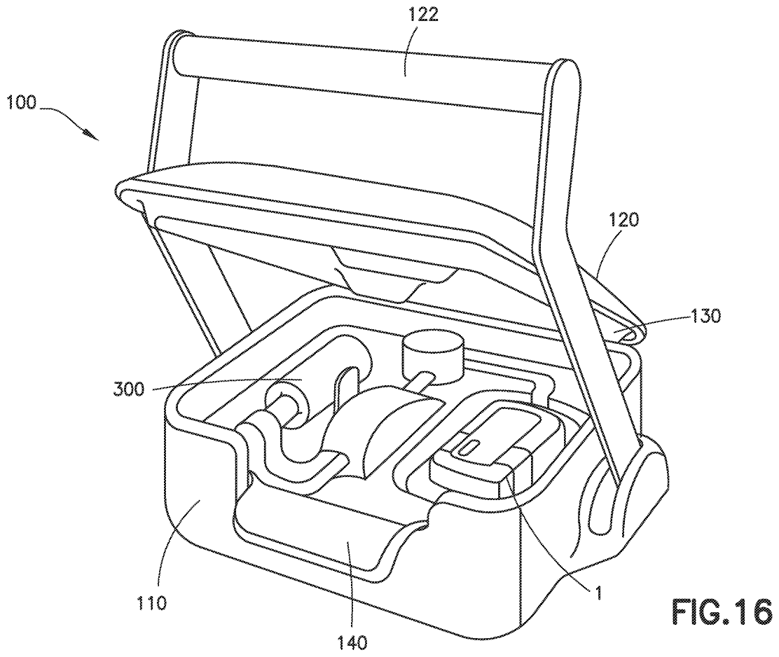

Referring now to FIG. 3, the drug delivery device 1 is shown with the main cover 2 and reservoir bag 4 removed so that the location of various other components can be appreciated. In some embodiments, a fill port 43 is provided in fluid communication with the reservoir bag 4 (not shown in FIG. 3) to facilitate filling of the reservoir bag 4 using a syringe or filling station 100 (shown in FIGS. 16-18). A receptacle 32 may be connected to the insertion mechanism 7 by tubing, for example, to transfer the medicament to the insertion mechanism 7 prior to injection into the skin of the patient.

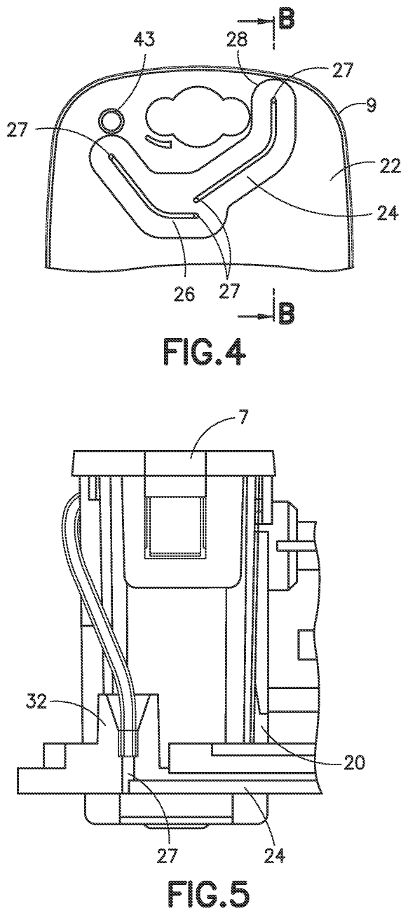

FIG. 4 illustrates a bottom surface 22 of the base 9 of the drug delivery device 1, prior to application of the adhesive pad 70 (shown in FIG. 1). During use, the bottom surface 22 is oriented toward the skin of the patient, such that the bottom surface 22 is neither visible nor readily accessible without disconnecting the drug delivery device 1 from the patient. As shown in FIG. 4, the bottom surface 22 of the base 9 may include first and second fluid channels 24, 26. The first and second fluid channels 24, 26 provide fluid pathways between various components such as the reservoir bag 4, the fill port 43, the force sensing resistor 30, the pump 3, and the insertion mechanism 7 (shown in, e.g., FIG. 2). The first and second fluid channels 24, 26 may be recessed from (or inscribed into) the bottom surface 22, and may be formed through a molding process, such as injection molding, or by a cutting process, such as milling. In other embodiments, the first and second fluid channels 24, 26 are disposed on the main cover 2, or on the base 9 within the interior 12 of the drug delivery device 1. The first and second fluid channels 24, 26 are sized such that flow rate of medicament is limited by the pump 3 and/or cannula 47 sizing, not by any flow restriction introduced by the first and second fluid channels 24, 26. In particular, the cross-sectional area of the first fluid channel 24 and the second fluid channel 26 may be greater than the gage of the cannula 47.

With continued reference to FIG. 4 and further reference to FIG. 5, the base 9 includes a fluid channel passageway 27 at each end of the first fluid channel 24 and the second fluid channel 26. As shown in FIG. 5, one of the fluid channel passageways 27 fluidly connects the receptacle 32 to a first end of the first fluid channel 24. Another of the fluid channel passageways 27 may fluidly connect the second end of the first fluid channel 27 to the pump 3. Similarly, one of the fluid channel passageways 27 may fluid connect a first end of the second fluid channel 26 to the fill port 43, and another of the fluid channel passageways 27 may fluidly connect a second end of the second fluid channel 26 to the pump 3.

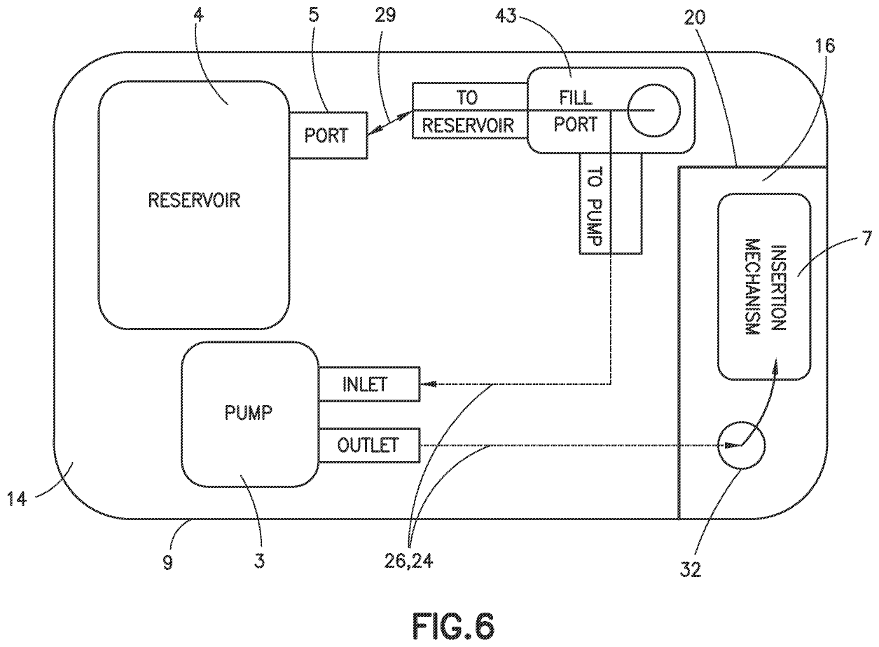

FIG. 6 is a schematic of an exemplary fluid path of the drug delivery device 1 in accordance with an embodiment of the present disclosure. During operation of the drug delivery device 1, the pump 3 draws medicament from the reservoir bag 4 via an inlet/outlet port 5 thereof, through a reservoir fluid channel 29 past the fill port 43, and into an inlet of the pump 3 via the second fluid channel 26. Next, the pump 3 drives the medicament into the first fluid channel 24, and subsequently to the receptacle 32 of the insertion mechanism 7. Finally, the insertion mechanism 7 receives the medicament from the receptacle 32 via tubing, for example, and delivers the medicament through the cannula 47 to the skin of the patient.

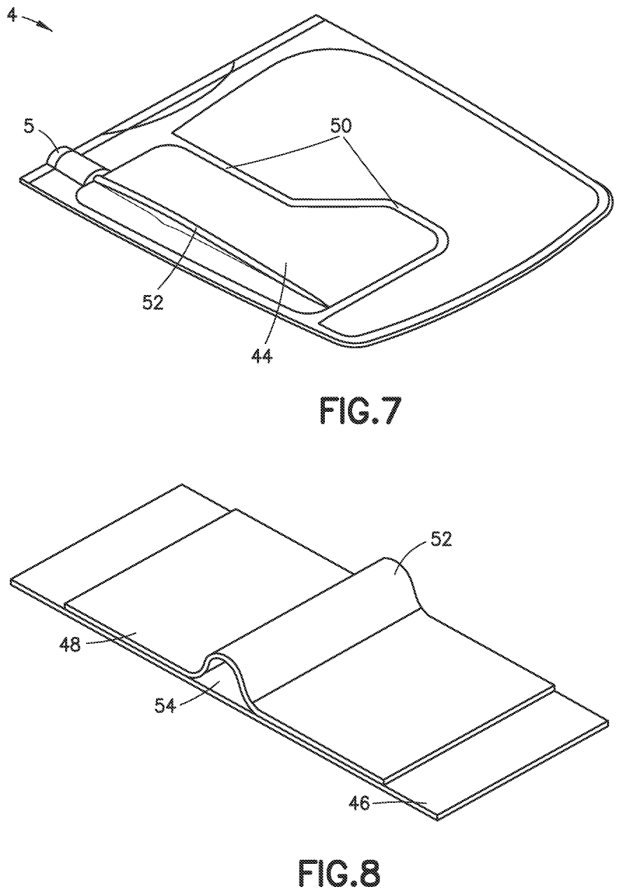

FIG. 7 shows the reservoir bag 4 separate from the drug delivery device 1 and in a substantially empty state. The reservoir bag 4 defines a medicament chamber 44 in fluid communication with an inlet/outlet port 5. The reservoir bag 4 may be constructed from compliant, flexible membranes joined along a border 50 to define the medicament chamber 44. The compliant, flexible membranes may include a first membrane layer 46 and a second membrane layer 48. The first and second membrane layers 46, 48 may be made of any suitable material, for example polyolefin/styrene block copolymer. The border 50 may be formed by heat sealing the first and second membrane layers 46, 48 to one another, for example in a press or mold.

In some embodiments, a tunnel 52 extends from the inlet/outlet port 5 at least partially into the medicament chamber 44. The tunnel 52 is defined in at least one of the first membrane layer 46 or the second membrane layer 48, and prevents the first and second membrane layers 46, 48 from laying entirely flat against one another. Thus, the tunnel 52 defines and maintains a flow channel 54 in fluid communication with the medicament chamber 44 and the inlet/outlet port 5. The tunnel 52 therefore prevents the medicament chamber 44 from self-sealing due to vacuum generated by the pump 3 pulling the first and second membrane layers 46, 48 against one another. In some embodiments, the tunnel 52 is sufficiently strong to maintain shape under vacuum produced by the pump 3 during normal operation of the drug delivery device 1. In some embodiments, the tunnel 52 extends an entire length of the medicament chamber 44, as shown in FIG. 7. In other embodiments, the tunnel 52 terminates at an intermediate location within the medicament chamber 44.

FIG. 8 shows a sectional view of the tunnel 52 with the medicament chamber 44 substantially empty, illustrating how the tunnel 52 prevents the first and second membrane layers 46, 48 from lying flat against one another in a manner that could obstruct flow to the inlet/outlet port 5. This is particularly evident with the medicament chamber 44 nearly empty, when the first and second membrane layers 46, 48 are most susceptible to interfacing with one another in a manner that could obstruct flow. The tunnel 52 may have a raised profile, such as an arched profile, as shown in FIG. 8, though other curved and polygonal profiles are understood to be within the scope of the present disclosure.

As shown in FIG. 8, the tunnel 52 may be formed in the second membrane layer 48, so a surface area of the second membrane layer 48 is greater than a surface area of the first membrane layer 46. Thus, the first membrane layer 46 is asymmetric relative to the second membrane layer 48. In other embodiments, the surface area of the second membrane layer 48 is approximately equal to the surface area of the first membrane layer 46. In some embodiments, the first membrane layer 46 may be symmetric relative to the second membrane layer 48.

In some embodiments, the tunnel 52 is not actively formed in the reservoir bag 4 during manufacturing; but is rather a consequence of the difference in surface area and the asymmetry of the first and second membrane layers 46, 48. That is, as the medicament reservoir is emptied by the pump 3, the greater surface area of the second membrane layer 48 is forced to develop one or more folds to take up the extra surface area of the second membrane layer 48 relative to the first membrane layer 46. These one or more folds from the tunnel 52.

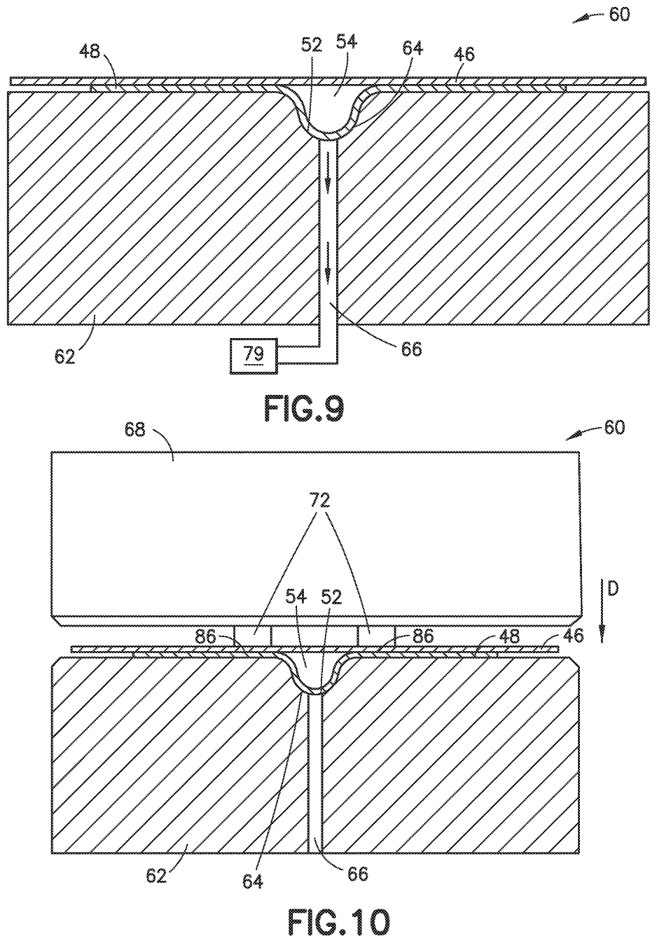

FIGS. 9-13 illustrate various embodiments of tooling for forming the tunnel 52 in the reservoir bag 4. Referring first to FIGS. 9 and 10, a vacuum molding device 60 for forming the tunnel 52 is illustrated. The vacuum molding device 60 includes a die 62 defining a cavity 64 corresponding to the profile of the tunnel 52. A vacuum channel 66 extends from the cavity 64 to an external vacuum source 79. The first and second membrane layers 46, 48 are positioned on the die 62 overlaying the cavity 64. A vacuum applied to the vacuum channel 66 draws the second membrane layer 48 into the cavity 64 to form the tunnel 52 in the second membrane layer 48. While the second membrane layer 48 is drawn into the cavity 64, the first membrane layer 46 remains stationary, resulting in asymmetry of the first and second membrane layers 46, 48.

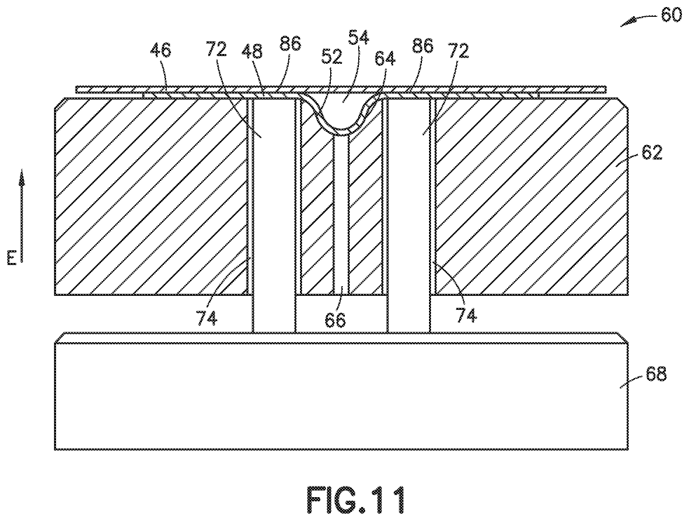

Referring now to FIGS. 10-11 the molding device 60 may include a sealing plate 68 to join the first membrane layer 46 to the second membrane layer 48 after the tunnel 52 has been formed. After being so joined, the first and second membrane layers 46, 48 cannot slide relative to one another, meaning the tunnel 52 is locked in place and cannot collapse. The sealing plate 68 includes one or more profiles 72, such as one or more prongs, which are heated by an external power source. The heated profiles 72 are brought into contact with the first and/or second membrane layers 46, 48 to create a spot weld 86 joining the first and second membrane layers 46, 48 to one another. FIG. 10 illustrates an embodiment of the molding device 60 in which the sealing plate 68 is positioned on the opposite side of the first and second membrane layers 46, 48 relative to the die 62. The sealing plate 68 is moved toward the die 62 in the direction of arrow D so that the one or more profiles 72 engage the first and/or second membrane layers 46, 48.

FIG. 11 illustrates an alternate embodiment in which the sealing plate 68 is positioned below the die 62 (i.e. on the opposite side of the die 62 relative to the first and second membrane layers 46, 48). The one or more profiles 72 (e.g. prongs) extend through corresponding passageways 74 in the die 62, allowing the profiles 72 to engage the first and/or second membrane layers 46, 48 as the sealing plate 68 is advanced toward the die 62 in the direction of arrow E. A backing plate to retain the first and second membrane layers 46, 48 against the die 62 is not shown.

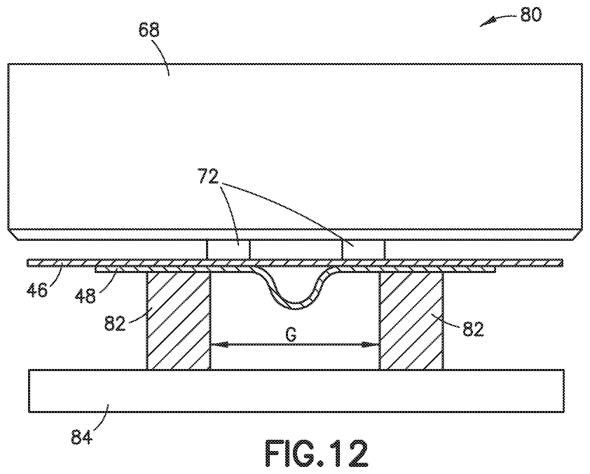

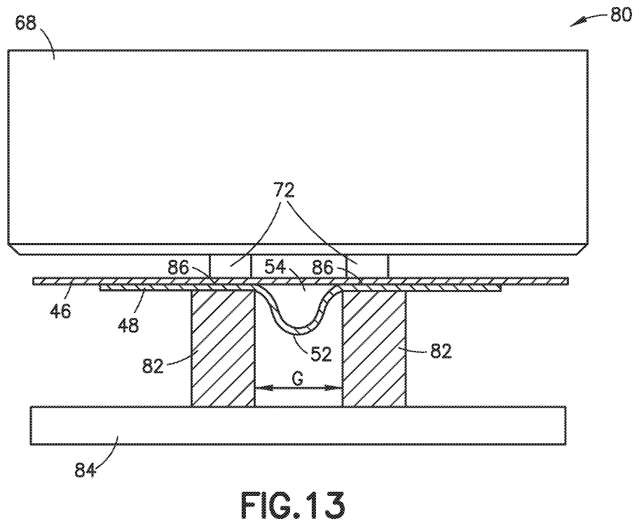

FIGS. 12 and 13 illustrate an alternative embodiment of tooling for forming the tunnel 52. A pinch-forming device 80 for forming the tunnel 52 includes a pair of pinching grips 82 that engage the second membrane layer 48 on opposite sides of the tunnel to be formed. The pair of pinching grips 82 are connected to an actuator 84, such as a cylinder, that draws the pinching grips 82 toward one another. Thus, a distance G between the pinching grips 82 decreases from a first initial position (shown in FIG. 12) to a second pinched position (shown in FIG. 13). The drawing together of the pinching grips 82 pinches the second membrane layer 48 to form the tunnel 52. The first membrane layer 46 remains stationary while the pinching grips 82 move between the first and second position to form the tunnel 52 in the second membrane layer 48. As with the embodiment of FIG. 10, the tooling may further include a sealing plate 68 with one or more profiles 72 to join the first and second membrane layers 46, 48 to lock the tunnel 52 in place.

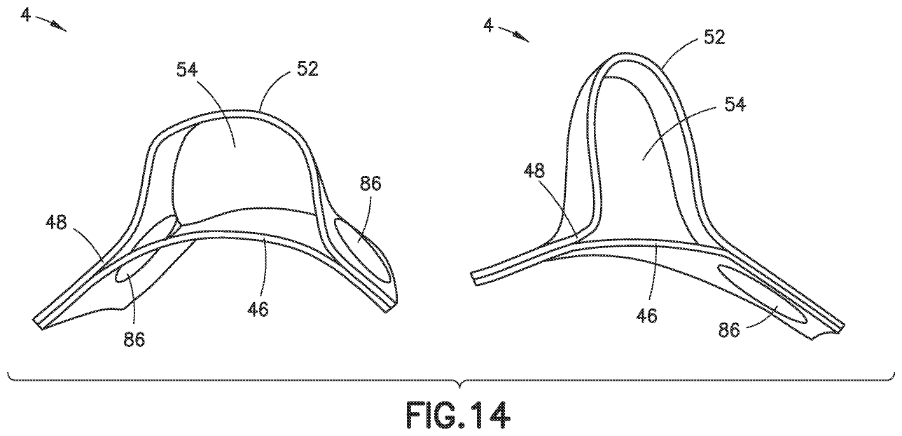

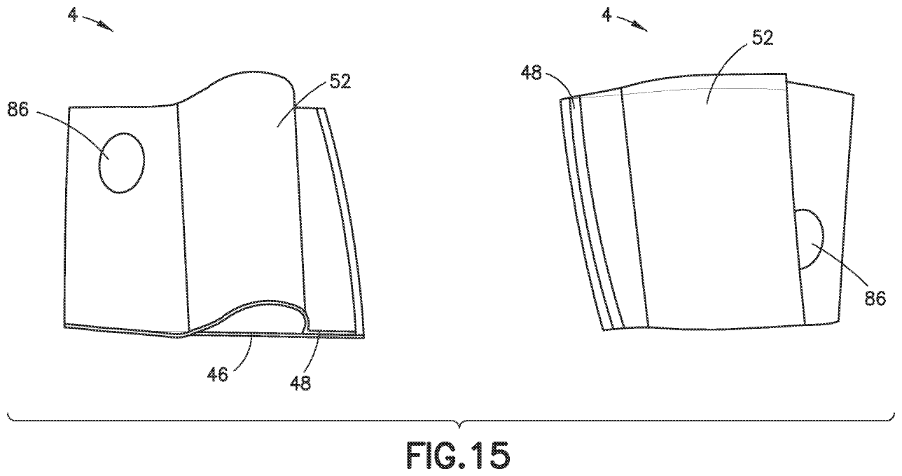

FIGS. 14 and 15 illustrate cross sections of the reservoir bag 4 with the tunnel 52 formed by the tooling of FIGS. 9-13. As discussed above, the reservoir bag 4 includes spot welds 86 where the one or more prongs 72 join the first membrane layer 46 to the second membrane layer 48.

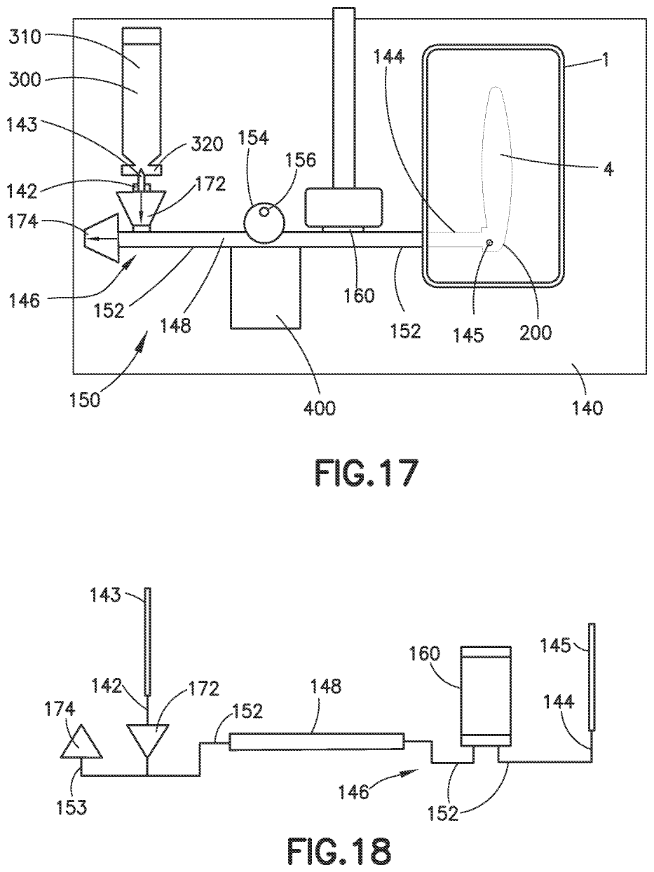

Referring now to FIGS. 16-18, in some embodiments, the reservoir bag 4 described in the present disclosure may be configured as a refill reservoir bag 300 for connection to an injector filling station 100 used to fill (and/or refill) one or more drug delivery devices 1. The refill reservoir bag 300 may include the same or similar features as the reservoir bag 4 described herein in connection with FIGS. 1-15. Referring now to FIG. 16, an embodiment of an injector filling station 100 is illustrated in accordance with an embodiment of the present disclosure. The filling station 100 can include a base 110 and a lid 120. The lid 120 can be pivotally connected to the base 110, for example by a rotational hinge 130. In some embodiments, the lid 120 includes a handle 122 to assist a user in manipulating the lid 120. The base 110 and/or the lid 120 can removably support a transfer tray 140. The tray 140 includes various features for supporting and removably retaining the drug delivery device 1 to filled, such as a snap-fit connector, bayonet connector, or the like. The tray 140 further includes various features for supporting and removably retaining the refill reservoir bag 300 containing medicament, such as a snap-fit connector, bayonet connector, or the like. The tray 140 includes various features for establishing fluid communication between the refill reservoir bag 300 and the reservoir bag 4 of the drug delivery device 1. In use, a user (typically a patient or the patient's caregiver) loads an empty drug delivery device 1 and the refill reservoir bag 300 onto the tray 140. The user then inserts the tray 140 into the base 110 and/or the lid 120 of the filling station 100. The tray 140, the base 110, and/or the lid 120 include various structures and components for transferring the medicament from the refill reservoir bag 300 into the reservoir of the drug delivery device 1, as will be described in connection with FIGS. 17 and 18. In some embodiments, transfer of the medicament from the refill reservoir bag 300 into the reservoir bag 4 of the drug delivery device 1 may occur automatically upon closure of the lid 120.

Referring now to FIGS. 17 and 18, the drug delivery device 1 and the refill reservoir bag 300 can be removably connected to the tray 140 such that a fluid path is established between a medicament chamber 310 (corresponding to the medicament chamber 44 shown in FIG. 7) of the refill reservoir bag 300 and the medicament chamber 44 (as shown in FIG. 7) of the reservoir bag 4 of the drug delivery device 1. The tray 140 includes a cartridge socket 142 for removably receiving the refill reservoir bag 300 and an injector socket 144 for removably receiving the drug delivery device 1. The tray 140 further includes a fluid path section 146 providing fluid communication between the cartridge socket 142 and the injector socket 144. As shown in FIG. 18, the cartridge socket 142 can include a needle 143 for piercing a septum or stopper 320 of the refill reservoir bag 300 to provide fluid communication between the fluid path section 146 and the medicament chamber 310 of the refill reservoir bag 300. Similarly, the injector socket 144 can include a needle 145 for piercing a septum or port 220 of the drug delivery device 1 to provide fluid communication between the fluid path section 146 and the medicament reservoir 210 of the drug delivery device 1. The fluid path section 146 can include one or more fluid channels 152 molded and/or formed into a base material 150 of the tray 140, and/or one or more tubing sections 148 connected to the base material 150.

With continued reference to FIG. 17, the tray 140 and/or other components of the filling station 100 include a pump 154 or other component for transferring medicament out of the refill reservoir bag 300 and into the reservoir bag 4 of the drug delivery device 1. The pump 154 can be disposed in the fluid path section 146. The pump 154 is controlled by an electronic controller 400 programmed or configured to actuate the pump 154 until a refill operation has been completed, and/or to abort the refill operation in the event of an error in loading or refilling the drug delivery device 1. In the embodiment shown in FIG. 17, the pump 154 is a peristaltic pump having one or more rollers 156 configured to engage a resilient tubing section 148 of the fluid path section 146 in order to draw medicament out of the refill reservoir bag 300 and inject the medicament into the reservoir bag 4 of the drug delivery device 1.

With continued reference to FIGS. 17 and 18, the tray 140 may further include an inflation chamber 160 configured to inject a pre-inflation volume of air into the drug delivery device 1 prior to refilling the drug delivery device 1 with medicament. In embodiments in which the drug delivery device 1 has a reservoir volume of approximately 3 milliliters (mL), the inflation chamber 160 may be configured to inject a pre-fill inflation volume of approximately 100 microliters (μL) of air per injector refill operation. The inflation chamber 160 is disposed in fluid communication with the fluid path section 146 between the cartridge socket 142 and the injector socket 144 such that the inflation chamber 160 supplies air to the drug delivery device 1 via the fluid path section 146. In the illustrated embodiment, the inflation chamber includes a piston 162 that moves axially to eject pressurized air from the inflation chamber 160.

With continued reference to FIGS. 17 and 18, the tray 140 can further include a forward check valve 172 disposed in the fluid path section 146 between the drug delivery device 1 and the refill reservoir bag 300. The forward check valve 172 prevents flow of air and/or medicament back from the drug delivery device 1 into the refill reservoir bag 300. The tray 140 can further include a reverse check valve 174 disposed in the fluid path section 146 to allow air to escape from the fluid path section 146. The reverse check valve 174 can be disposed in a branch channel 153 of the fluid path section 146 arranged such that fluid can flow from the drug delivery device 1 to the reverse check valve 174 without flowing into the refill reservoir bag 300, and such that fluid can flow from the refill reservoir bag 300 to the reverse check valve 174 without flowing into the drug delivery device 1. In other words, the drug delivery device 1 and the refill reservoir bag 300 are in parallel fluid circuits relative to the reverse check valve 174.

In some embodiments, the cartridge socket 142 can include a port with which the refill reservoir bag 300 has a snap-fit connection. In other embodiments, the cartridge socket 142 can include a bayonet connector, a threaded fitting, or other secure mechanical connection to engage and hold the refill reservoir bag 300. The cartridge socket 142 may exhibit tactile feedback, such as a snap fit, to confirm to the user that the refill reservoir bag 300 is properly inserted into the cartridge socket 142. Connection of the refill reservoir bag 300 to the cartridge socket 142 can automatically cause the needle 143 of the cartridge socket 142 to pierce the septum or stopper (which may, for example, be includes in the inlet/outlet port 5 as shown in FIG. 7) of the refill reservoir bag 300. Likewise, the injector socket 144 can include a port with which the fill port 43 of the drug delivery device 1 has a snap-fit connection. The refill socket 142 may exhibit tactile feedback, such as a snap fit, to confirm to the user that the drug delivery device 1 is properly inserted into the injector socket 144. Connection of the drug delivery device 1 to the injector socket 144 can automatically cause the needle 145 of the injector socket 144 to pierce the septum of the drug delivery device 1.

Through a variety of mechanical and/or electromechanical components, the pump 154 may be automatically actuated when the handle 122 of the filling station 100 is manipulated to close the lid 120. As explained above, the pump 154 then transfers medicament from the refill reservoir bag 300 to the reservoir bag 4 of the drug delivery device 1. The handle 122 may subsequently be raised to open the lid 120, and the refilled drug delivery device 1 can be removed and put into use.

Although the disclosure has been described in detail for the purpose of illustration based on what is currently considered to be the most practical and preferred embodiments, it is to be understood that such detail is solely for that purpose and that the disclosure is not limited to the disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

1. A drug delivery device configured to deliver a medicament to a patient, the drug delivery device comprising:
a cannula for piercing the patient's skin;
a pump for driving medicament to the cannula; and
a reservoir bag fluidly connected to the pump, the reservoir bag comprising a first membrane layer and a second membrane layer defining a medicament chamber,
wherein at least one of the first membrane layer and the second membrane layer comprises a tunnel extending at least partially into the medicament chamber.

2. The drug delivery device of claim 1, wherein the reservoir bag is an internal reservoir bag contained within an internal region of the drug delivery device.

3. The drug delivery device of claim 1, wherein the first membrane layer is asymmetric relative to the second membrane layer.

4. The drug delivery device of claim 1, wherein the first membrane layer is symmetric relative to the second membrane layer.

5. The drug delivery device of claim 1, wherein a surface area of the second membrane layer is greater than a surface area of the first membrane layer.

6. The drug delivery device of claim 1, wherein a surface area of the second membrane layer is approximately the same as a surface area of the first membrane layer.

7. The drug delivery device of claim 1, wherein the tunnel has a raised profile.

8. The drug delivery device of claim 1, wherein the tunnel defines a flow channel in fluid communication with a port of the reservoir bag.

9. The drug delivery device of claim 1, wherein the tunnel extends an entire length of the medicament chamber.

10. The drug delivery device of claim 1, wherein the tunnel terminates at an intermediate location within the medicament chamber.

11. The drug delivery device of claim 1, wherein the tunnel is sufficiently strong to maintain shape under vacuum produced by the pump.

12. The drug delivery device of claim 1, wherein the tunnel prevents the first and second membrane layers from laying entirely flat against one another.

13. A vacuum molding device for forming a reservoir bag of a drug delivery device, the vacuum molding device comprising:
a die defining a cavity; and
a vacuum channel extending from the cavity,
wherein the cavity corresponds to a profile of a tunnel to be formed in the internal reservoir bag, and
wherein the vacuum channel is configured to draw at least one of a first membrane layer and a second membrane layer of the reservoir bag into the cavity.

14. The vacuum molding device of claim 13, further comprising:
a sealing plate configured to join the first membrane layer to the second membrane layer.

15. The vacuum molding device of claim 14, wherein the sealing plate comprises one or more prongs configured to join the first membrane layer to the second membrane layer.

16. The vacuum molding device of claim 14, wherein the sealing plate is configured to be moved toward the die.

17. The vacuum molding device of claim 14, wherein the die comprises one or more passageways through which one or more profiles of the sealing plate extend.

18. A pinch-forming device for forming a reservoir bag of a drug delivery device, the pinch-forming device comprising:
a pair of pinching grips configured for engaging a second membrane layer of the reservoir bag on opposite sides of a tunnel to be formed in the second membrane layer; and
an actuator configured for drawing the pinching grips toward one another to form the tunnel in the second membrane layer,
wherein a first membrane layer of the reservoir bag remains stationary as the actuator draws the pinching grips toward one another.

19. The pinch-forming device of claim 18, wherein the actuator comprises a cylinder.

20. The pinch-forming device of claim 18, further comprising:
a sealing plate configured to join the first membrane layer to the second membrane layer, the sealing plate comprises one or more prongs configured to join the first membrane layer to the second membrane layer.

21.-46. (canceled)
